(19) United States
(12) Patent Application Publication
(10) Pub. No.: US 2008/0015367 A1
(43) Pub. Date: Jan. 17, 2008

(54) PROCESS FOR ISOLATING PHYTOSTEROLS AND TOCOPHEROLS FROM DEODORIZER DISTILLATE

(75) Inventors: Thomas A. Dobbins, Howard, OH (US); David B. Wiley, Coshocton, OH (US); Deborah C. Dobbins, Howard, OH (US)

Correspondence Address: THOMPSON HINE L.L.P.;Intellectual Property Group, P.O. BOX 8801, DAYTON, OH, 45401-8801, US

(73) Assignee: WILEY ORGANICS, INC., Coshocton, OH (US)

(21) Appl. No.: 11/776,123

(22) Filed: Jul. 11, 2007

Related U.S. Application Data
(60) Provisional application No. 60/819,848, filed on Jul. 11, 2006.

(51) Int. Cl. C07D 311/72; C07J 75/00

(52) U.S. Cl. 549/407; 552/502

(57) ABSTRACT

A process is described for efficiently recovering at least one of phytosterols and tocopherols in substantially pure form from the deodorizer distillates that may be obtained as by-products of the refining of edible oils. The phytosterol fatty acid esters present in deodorizer distillate are saponified with potassium hydroxide in a solvent medium containing methanol and water, thereby forming a solvent medium containing methanol, water, and the potassium soaps of fatty acids. Unsaponiable matter including tocopherols remain dissolved in this medium, allowing recovery of substantially pure phytosterols. Tocopherols in substantially pure form may be recovered by distillation under reduced pressure.

### CROSS REFERENCE TO RELATED APPLICATION

This application claims the benefit of U.S. Provisional Application No. 60/819,848, filed Jul. 11, 2006, the contents of which are hereby incorporated by reference.

### BACKGROUND

Phytosterols (also known as “plant sterols”) are a group of steroid alcohol phytochemicals that are abundant in nature, occurring naturally in a variety of fruits and vegetables that are part of the human diet. In plants phytosterols act as a structural component in cell membranes, filling the role that is played by cholesterol in mammalian cells. In pure form, phytosterols are white powders that are insoluble in water, only moderately soluble in lower aliphatic alcohols and ketones, but quite soluble in hydrocarbons and other non-polar organic solvents such as ethers.

Recovering phytosterols to make value-added products is important from a commercial point of view. Phytosterols have found applications as starting materials in the synthesis of steroidal drugs and pesticides, as emulsifiers in cosmetics, and as nutritional supplements and food additives. Phytosterols cannot be synthesized in the human body and are obtained exclusively through the diet. Vegetable oils are an excellent source of phytosterols.

A second class of compounds found in vegetable oils (especially soybean, sunflower, corn, olive, and palm oils) is known as tocopherols. In pure form, tocopherols are high-boiling liquids that are insoluble in water, only sparingly soluble in lower aliphatic alcohols and ketones, and highly soluble or miscible in hydrocarbons and other non-polar organic solvents such as ethers. Tocopherols have important commercial applications as nutritional supplements (Vitamin E), food additives, and anti-oxidants.

One of the principal commercial sources of both phytosterols and tocopherols is “deodorizer distillate,” a by-product of refining edible oils. The isolation of phytosterols and tocopherols from deodorizer distillate is industrially very important because of the immense quantities of vegetable oils that are refined annually. One of the final steps in refining vegetable oils to produce edible oils with bland flavor and odor and good shelf life involves the removal of oxidation products (largely aldehydes). This is achieved by heating the oil to temperatures of 230° to 250° C. under reduced pressure (typically 2 to 15 mm Hg) while sparging steam below the surface of the hot oil, usually for a period of 60 to 90 minutes. Known as “steam deodorization,” this physical refining step produces a water-immiscible volatile fraction variously known as “deodorizer distillate” (“DOD”), “deodorizer sludge,” or “vegetable oil distillate” (“VOD”) that is separated from the steam by means of traps or condensers. (The preparation of deodorizer distillates and the apparatus for this purpose have been widely described in the technical literature, notably in the article “Deodorization and Finished Oil Handling” by Arnold M. Gavin in The Journal of the American Oil Chemists Society, Volume 58, pp. 175-184, March 1981.)

Deodorizer distillates are complex mixtures containing: free fatty acids; the monoglyceride, diglyceride, and triglyceride esters of the fatty acids present in the oil (predominantly the mono- and diglyceride esters); terpenic and aliphatic alcohols; waxes; squalene; carotenoid pigments; free phytosterols and phytosterol esters of fatty acids; and tocopherols.

The acid number (also known as the “acid value” or “neutralization number”) is the mass of potassium hydroxide measured in milligrams required to neutralize the free acids in one gram of a substance. The acid number is a measure of the amount of free carboxylic acids (i.e. fatty acids) present. Typically, the acid number of deodorizer distillates ranges from about 60 to 70, corresponding to a free fatty acid content of 20% to 27% by weight.

The “saponification number” (also known as the “saponification value”) corresponds to the number of milligrams of potassium hydroxide required to saponify the esters contained in one gram of a substance. Typically, the saponification number of deodorizer distillates ranges from about 150 to 180. The fact that the saponification number of deodorizer distillate is invariably higher than the acid number is attributable to the presence of mono-, di-, and triglyceride esters of free fatty acids and other fatty acid esters, notably phytosterol esters.

Although phytosterols and tocopherols are only trace constituents (0.01% to 0.9% by weight) of crude vegetable oils, they are very efficiently concentrated in deodorizer distillates by the steam deodorizing refining process. Depending upon the botanical source and processing conditions (i.e. temperature, pressure, steam quantity, duration of steam sparging), the phytosterol content and the tocopherol content of deodorizer distillates vary widely, but typically fall in the range of 10% to 24% by weight for phytosterols and 6% to 12% for tocopherols in deodorizer distillates derived from soybean oil.

Phytosterols occur in deodorizer distillates both in the free form and as fatty acid esters. The amount of phytosterol esters in deodorizer distillates usually exceeds the amount of free phytosterols. In many deodorizer distillates, the ratio of phytosterol esters to free phytosterols is as great as 2:1. The thermal history of the deodorizer distillate during processing plays a major role in determining this ratio. U.S. Pat. No. 5,487,817 teaches that free phytosterols react with the free fatty acids present in deodorizer distillates to form esters simply by heating the deodorizer distillates to temperatures exceeding 150° C. for several hours.

Most commercial applications require phytosterols of high purity (greater than 90%). The direct isolation of phytosterols or tocopherols from deodorizer distillates by fractional distillation under reduced pressure is not a practical approach for several reasons:
1.) The boiling points of phytosterols and other unsaponifiable materials found in deodorizer distillates (notably tocopherols) do not differ greatly, making it intrinsically difficult to obtain substantially pure phytosterols (or substantially pure tocopherols) by distillation. The phytosterol-to-tocopherol ratios of deodorizer distillates typically range from 1:1 to 3:1, depending on the vegetable oil source. So any distillate containing both the tocopherols and sterols must be subjected to additional separation techniques in order to produce a phytosterol concentrate that is essentially free of tocopherols or a tocopherol concentrate that is essentially free of phytosterols. U.S. Pat. No. 4,454,329, for example, succeeds only in isolating a concentrate containing phytosterols, tocopherols, and various other compounds with similar boiling points in a distillate derived from an esterified deodorizer distillate.
2.) When subjected to prolonged exposure to the temperatures required for distillation (typically in excess of 200° C.), phytosterols are prone to undergo irreversible dehydration and condensation reactions to form compounds known as steradienes and disteryl ethers, respectively.
3.) At the temperatures required for distillation, an appreciable fraction of the phytosterols reacts with the free fatty acids that are present in abundance in deodorizer distillates to form phytosterol esters that are far less volatile than free phytosterols.
4.) Finally, because a substantial portion of the phytosterols present in deodorizer distillates already occur in the form of fatty acid esters, the overall recovery of phytosterols is poor unless the phytosterol esters are saponified or transesterified to liberate free phytosterols.

Consequently, all commercially viable processes for isolating sterols from deodorizer distillates involve a preliminary step to convert the phytosterol fatty acid esters to free phytosterols. This may be accomplished in one of three ways:
1.) By microbial hydrolysis of the phytosterol ester via lipase enzymes.
2.) By means of a transesterification reaction with an aliphatic alcohol of low molecular weight such as methanol, usually catalyzed by an alkali metal alkoxide under anhydrous conditions. Free sterols are liberated while forming an ester of markedly greater volatility that can be separated by distillation or crystallization.
3.) By hydrolysis under strongly basic conditions with an alkali like sodium or potassium hydroxide (i.e. saponification).

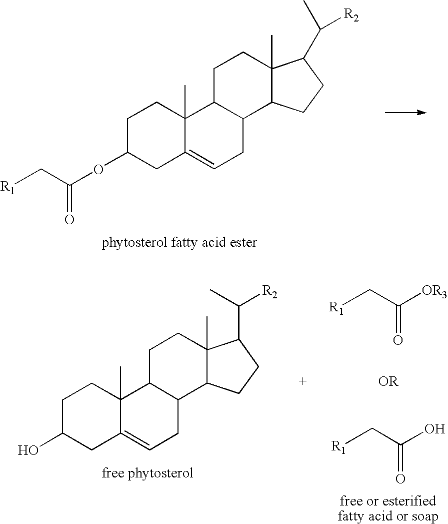

Of the three methods for liberating free phytosterols from phytosterol esters, the simplest to practice and by far the most widely employed on a commercial scale is saponification. Following the saponification reaction, the free sterols must be separated from both the soaps and from the other unsaponifiable matter such as glycerol, squalene, waxes, pigments, and tocopherols.

A recent approach to effecting the separation of phytosterols from soaps and unsaponifiables is fractional distillation under reduced pressure. The soaps, being non-volatile, remain in the distillation heel. Despite the large capital investment required to practice this method, the phytosterols are not isolated in a substantially pure form in the distillate and the phytosterol-enriched distillate fractions obtained must be further purified by crystallization from one or more solvents.

The traditional approach to effecting the separation of phytosterols from the mixtures of soaps and unsaponifiables that result from saponifying deodorizer distillates is by a series of successive extractions with a variety of solvents. In general, these methods for recovering phytosterols from saponified deodorizer distillate consist of sequestering the soaps in an aqueous or polar, hydrophilic solvent while the phytosterols and other unsaponifiable matter are extracted into a second immiscible solvent of lesser polarity.

While the unsaponifiable fractions are readily soluble in water-immiscible solvents in general and hydrocarbons and halogenated hydrocarbons in particular, it is seldom possible to isolate substantially pure phytosterols from such merely by concentrating the solution and performing fractional crystallization. The fraction rich in phytosterols obtained in this fashion is always too contaminated by other unsaponifiable components like tocopherols that are also present in the solution. For this reason, it is normally necessary to further purify the phytosterols by repeated recrystallizations from one or more solvents, especially alcohols or ketones, taking advantage of the relatively low solubility of phytosterols in solvents like lower aliphatic alcohols and ketones compared with the solubility of tocopherols, squalene, and other unsaponifiable matter in such solvents. In industrial processing this often leads to expensive process design as well as poor yields.

One process for the isolation of sterols from vegetable-derived lipids is described in U.S. Pat. No. 2,866,797. In this process, the fatty components of the vegetable source are saponified with an aqueous solution of a strong base such as sodium hydroxide. The aqueous solution of the saponification products is then extracted with a water-immiscible chlorinated hydrocarbon such as dichloroethylene. The sodium soaps remain in the aqueous phase, while the phytosterols and other unsaponifiable species (including tocopherols) dissolve in the immiscible hydrocarbon phase, which is decanted or otherwise isolated. Modest amounts of water and a water-miscible polar organic solvent like methanol or ethanol are then added to the extract. The resulting mixture is then cooled in order to separate the solid sterols from the other unsaponifiable species such as tocopherols. This process is seldom practiced on a commercial scale because the chlorinated hydrocarbon solvents employed have adverse environmental impacts, and the purity of the recovered sterols is only moderate unless subsequent crystallization or other processing steps are employed.

U.S. Pat. No. 4,044,031 discloses a process in which the unsaponifiable fraction is extracted from a saponification reaction mixture into a suitable water-immiscible solvent such as hexane, followed by a second extraction of the phytosterols and other polar components from this solution into a methanol-acetone mixture to which a small amount of water is added in order to affect phase separation. The wetted methanol-acetone solution is concentrated by evaporation. Upon cooling, substantially pure phytosterols crystallize out and are recovered by filtration or centrifugation.

In order to reduce the number of solvents required to isolate phytosterols from deodorizer distillate, U.S. Pat. No. 2,349,270 teaches a process whereby slaked lime (calcium hydroxide) is added directly to the deodorizer distillate, resulting in the saponification of the esters and the formation of a mass of insoluble solid calcium soaps. The mass of calcium soaps is broken up and the phytosterols, tocopherols, and other unsaponifiable matter are extracted into diethyl ether.

A similar approach is taught by U.S. Pat. No. 2,263,550. This patent discloses the more rapid saponification using sodium or potassium hydroxide, followed by the addition of calcium chloride to form insoluble calcium soaps. The calcium soaps are then extracted with acetone in order to separate the phytosterols, tocopherols, and other unsaponifiable matter.

U.S. Pat. No. 3,108,120 attempts to improve the “lime soap” processes disclosed in the two aforementioned patents. Noting that mixtures of calcium soaps and unsaponifiable matter tend to form hard, wax-like gels that are difficult to leach with solvents and that require heavy grinding or masticating machinery to subdivide into an extractable form, this patent teaches the addition of a finely divided, inert “powdering agent” to render the lime soap mass more amenable to extraction.

Like the preceding two patents cited above, U.S. Pat. No. 5,371,245 teaches saponification of deodorizer distillates using an alkali metal hydroxide (sodium or potassium hydroxide) in an alcoholic solvent, then adding a zinc halide salt to precipitate the fatty acids in the form of insoluble granular zinc soaps of uniform particle size that are readily removable by filtration (unlike the corresponding calcium fatty acid soaps), leaving dissolved glycerol, phytosterols, tocopherols, and other unsaponifiable species in the filtrate. The filtrate is concentrated by evaporation and subjected to solvent partitioning. The glycerols are sequestered in an aqueous medium, while the phytosterols and tocopherols are dissolved in a non-polar aliphatic hydrocarbon solvent. The separation of phytosterols from the tocopherols is poor, and neither is obtained in a substantially pure form without recourse to further processing steps.

It would be desirable to have a more efficient process for recovering valuable components, such as phytosterols and tocopherols, in substantially pure form from deodorizer distillates.

### SUMMARY OF THE INVENTION

The present invention relates to an improved process for recovering phytosterols and/or tocopherols from deodorizer distillates. In accordance with one aspect of the invention, phytosterols are recovered from a deodorizer distillate composition containing fatty acid esters of phytosterols by contacting the deodorizer distillate composition with methanol and potassium hydroxide to saponify the fatty acid esters in the deodorizer distillate composition and then adding water to form a mixture comprising a precipitate containing the phytosterols.

In accordance with another aspect of the invention, phytosterols and/or tocopherols are recovered from a deodorizer distillate composition by saponifying the phytosterol esters contained in the deodorizer distillate by contacting the composition with methanol and potassium hydroxide and adding water to the saponification reaction mixture to form a solvent composed of methanol, water and the potassium soaps of the fatty acids wherein the solvent causes precipitation of the phytosterols while dissolving or rendering miscible the tocopherols or other unsaponifiable matter in the saponification reaction mixture. The phytosterols can be recovered by filtration or centrifugation. The tocopherols can be recovered by acidifying the centrate or filtrate with a dilute aqueous solution of a mineral acid, separating the water-immiscible mixture of free fatty acids, tocopherols and other unsaponifiable matter and then isolating the tocopherols from the water-immiscible mixture of free fatty acids and unsaponifiable matter by fractional distillation under reduced pressure.

### BRIEF DESCRIPTION OF THE DRAWINGS

FIG. 1 is a flow chart describing the process for recovering phytosterols and tocopherols in accordance with one aspect of the present invention; and

FIG. 2 comprises FIGS. 2A AND 2B and is a flow chart describing the process for recovering phytosterols and tocopherols in accordance with another aspect of the present invention wherein the free fatty acids in the deodorizer distillate are converted to methyl esters and removed prior to saponification.

### DETAILED DESCRIPTION

The present invention relates to a process for efficiently recovering at least one of phytosterols and tocopherols from deodorizer distillates that may be obtained as by-products of the refining of edible oils. In accordance with one aspect of the invention, the phytosterol fatty acid esters present in deodorizer distillate are saponified with potassium hydroxide in a solvent medium containing methanol and water, forming a solvent medium containing methanol, water, and the potassium soaps of fatty acids. Unsaponifiable matter including tocopherols remain dissolved in this medium, allowing recovery of substantially pure phytosterols. Tocopherols may be recovered from the filtrate by distillation under reduced pressure.

Conventional methods for recovering phytosterols and tocopherols typically involved formation of insoluble soaps of calcium or zinc during or following saponification. The present invention takes the opposite approach. Rather than attempting to form insoluble soaps of calcium or zinc during or following saponification, the fatty acid moieties (occurring as either free fatty acids or as esters) are converted to soluble potassium soaps that remain dissolved in the saponification reaction mixture. Although not wishing to be bound by theory, the presence of these potassium soaps is thought to modify the properties of the polar alcoholic solvent employed in such a fashion that the unsaponifiable species that must be separated from the phytosterols such as tocopherols, squalene, carotenoids, and glycerol all remain in solution after the phytosterols crystallize out, permitting the phytosterols to be recovered in a substantially pure form and in high yield by filtration or centrifugation. In accordance with a particular aspect of the present invention, the phytosterols are separated from both the soaps and from tocopherols and other unsaponifiables in a single step from a single solvent medium rather than in multiple steps employing multiple solvents.

The potassium soaps of fatty acids are far more soluble in water and in polar organic solvents, especially lower aliphatic alcohols, than their sodium, calcium, or zinc counterparts. Although sodium hydroxide is significantly less expensive than potassium hydroxide and is an effective saponification agent, sodium soaps are not sufficiently soluble to be useful in practicing the present invention. Therefore, the present invention employs potassium hydroxide to effect the saponification of the esters present in deodorizer distillates, resulting in the formation of potassium soaps. The amount of potassium hydroxide employed is generally a modest (5% to 10%) stoichiometric excess based on the saponification number of the deodorizer distillate. Potassium hydroxide amounts outside this range may also be used but may not be as efficient. In one embodiment of the present invention, the solvent medium is a lower aliphatic alcohol (e.g. C₁ to C₃) containing a modest amount of water. Examples of useful lower aliphatic alcohols include but are not limited to methanol, ethanol, and isopropanol. In the preferred embodiment of the present invention, the best results are obtained with a solvent consisting of methanol containing no less than 5% and no more than 25% water by weight. Methanol and water mixtures outside these ranges may also be used but may not be as effective.

The solubility of the potassium soaps of fatty acids in mixtures of lower aliphatic alcohols and water varies. The potassium soaps of mono-unsaturated fatty acids such as oleic acid, and poly-unsaturated fatty acids like linoleic acid and linolenic acid, are significantly more soluble than the potassium soaps of saturated fatty acids like stearic acid. Because the process of the present invention requires the potassium soaps to remain dissolved in the wet alcoholic solvent, deodorizer distillates derived from some botanical sources give better results (i.e. higher purity and/or recovery of phytosterols and/or tocopherols) than others when subjected to the process of the present invention.

The fatty acid profiles of the edible oils depicted in the table below correspond closely to the fatty acid profiles of the deodorizer distillates obtained when those oils are refined. The process of the present invention has been found to give optimal results with deodorizer distillates derived from soybean oil, canola oil, and sunflower oil, which are comparatively low in saturated fatty acids. Deodorizer distillates derived from the refining of cottonseed oil, coconut oil, and palm oil yield poorer results due to the high saturated fatty acid content of these oils, as do deodorizer distillates obtained from soybean and other oils that have been “hardened” by hydrogenation to increase their saturated fatty acid content. As a rule of thumb, the best results are obtained with deodorizer distillates containing less than 30% saturated fatty acids as a percentage of the total fatty acids present, either in free form or as esters.

| | | MONO- | POLY- |
|---|---|---|---|
| OIL | SATURATED | UNSATURATED | UNSATURATED |
| Olive | 13.2 | 72.5 | 8.5 |
| Corn | 12.7 | 24.2 | 58.7 |
| Safflower | 6.2 | 74.6 | 14.4 |
| Canola | 5.8 | 56.1 | 29.6 |
| Soybean | 14.2 | 22.8 | 57.8 |
| Sesame | 13.7 | 39.3 | 41.6 |
| Sunflower | 8.9 | 45.3 | 40.0 |
| Flax Seed | 10.0 | 18.0 | 70.0 |
| Cottonseed | 25.8 | 17.0 | 51.7 |
| Coconut | 69.7 | 5.8 | 1.8 |
| Palm | 48.8 | 36.6 | 9.3 |

The following description refers to the use of methanol and water as the solvent medium, but as noted above other alcohols can be employed. The water may be present during the saponification reaction or it may be added following the completion of the saponification reaction. In the latter case, the saponification reaction will proceed more rapidly. The presence of water in the resulting methanolic solution of potassium soaps has two countervailing effects. Water increases the solubility of the potassium soaps while simultaneously decreasing the solubility of the unsaponifiable compounds such as tocopherols and squalene. In order to maximize the purity of the phytosterols that crystallize out of the methanol-water-potassium soap solvent upon cooling, it is desirable to keep both the potassium soaps and the unsaponifiable species such as tocopherols in solution.

The unsaponifiable compounds that must be separated from the phytosterols (such as tocopherols, squalene, waxes, and pigments) are insoluble or only very sparingly soluble in methanol or wet methanol. However, the presence of the dissolved potassium soaps produced by the saponification reaction has the surprising effect of rendering these materials soluble or miscible in the alcoholic solvent medium. In effect, the phytosterols are recovered by allowing them to crystallize or precipitate from a solvent that comprises a mixture of methanol, water, and potassium soaps of fatty acids in the proper proportions.

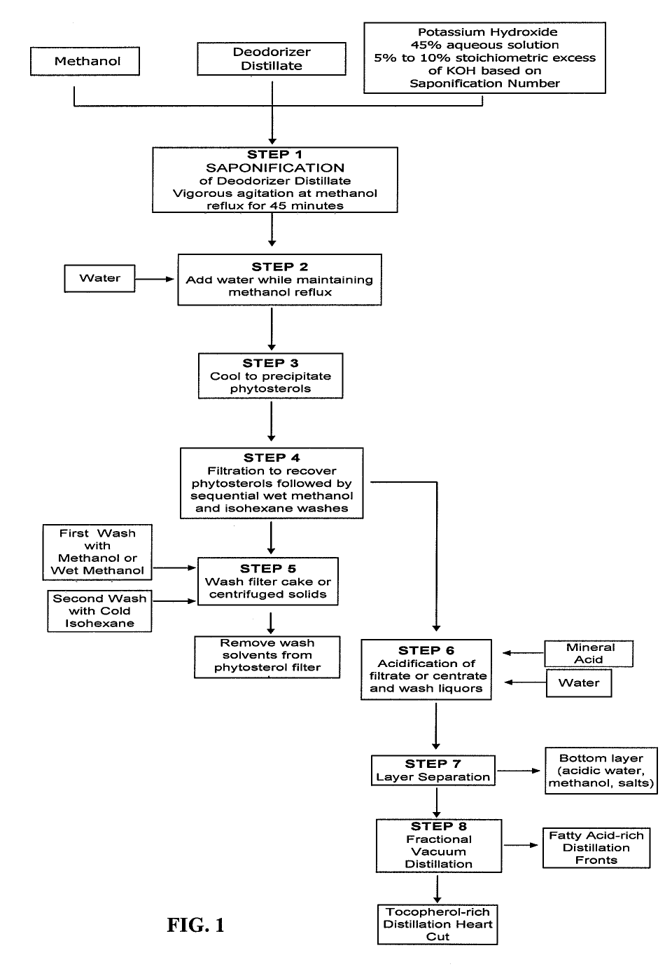

A particular process in accordance with one aspect of the present invention is described in the flow chart set out in FIG. 1. The described process includes the following steps:
1.) Digesting deodorizer distillate at an elevated temperature in its mass of methanol containing sufficient dissolved potassium hydroxide to effect the saponification of the esters contained in said deodorizer distillate. Typically, the amount of methanol used is from about one to about five times the mass of the deodorizer distillate. The potassium hydroxide may be added as a solid (flakes or pastilles) or, more conveniently, as a 45% by weight aqueous solution, which is an article of commerce. The amount of potassium hydroxide typically is a 5% to 10% stoichiometric excess based on the requirement determined by the saponification number of the deodorizer distillate, which typically falls in the range of 150 to 180 milligrams of potassium hydroxide per gram of deodorizer distillate. In methanol refluxing at atmospheric pressure (65° C.) with vigorous agitation, the deodorizer distillate will disperse rapidly and the saponification reaction will typically be completed in less than one hour. The saponification reaction typically is conducted at a temperature from about 50° C. to about 160° C. Elevated temperatures and pressures may be employed to further speed the saponification reaction. As the reaction proceeds, the formation of crystals of insoluble phytosterols will be observed.
2.) Adding the requisite amount of water to the saponification reaction mixture. Once the saponification reaction is complete, the pH of the mixture will exceed 10.5 and the methanol will contain the following solutes: the potassium soaps of fatty acids, glycerol liberated by the saponification of mono-, di-, and triglyceride fatty acid esters, and the unsaponifiable matter that includes terpenic and aliphatic alcohols, waxes, squalene, tocopherols, and pigments. A portion of the free phytosterols will also be in solution. The addition of the proper amount of water, typically about 5% to 30%, more particularly from about 16% to about 25% by weight of the methanol, will promote the continued solubility of the potassium soaps at lower temperatures and promote the precipitation of the phytosterols while maintaining the solubility of the other unsaponifiable species, notably tocopherols. In accordance with particularly useful aspects of the invention, the amount of the potassium soaps of fatty acids in the solvent typically is in the range of about 4% to 25% by weight based on the total weight of the solvent.
3.) Cooling the mixture to a temperature of 15° C. to 30° C., whereupon additional phytosterols will crystallize or precipitate.
4.) Filtering or centrifuging the cooled mixture to remove the phytosterols. When practiced on an industrial scale, a rotary drum filter gives excellent results.
5.) Washing the filter cake or centrifuged solids. When the filter cake is washed with a modest amount of methanol or wet methanol, the phytosterol product will typically have a purity of more than about 85%, and in particular cases, a purity of 88% to 94%. In accordance with particularly useful aspects of the invention, the ratio of water to methanol in the wash solution typically ranges from about 5% to 25% by weight. A second wash with a modest amount of non-polar aliphatic solvent such as but not limited to pentanes, hexanes, or heptanes typically increases the purity to more than about 90%, more specifically the purity is within the range from about 95% to 98%. Isohexane is ideal for this purpose because, unlike ordinary non-polar solvents such as n-hexane or n-heptane, it is not repelled by methanol or moderately wet methanol, keeping the filter cake highly permeable and free of any proclivity to “blind.” The non-polar washing solution may be used at temperatures less than ambient temperature, more particularly less than about 5° C.
6.) Acidifying the filtrate or centrate, which consists of methanol, water, and any isohexane or other non-polar wash solvent, the potassium soaps of fatty acids, and dissolved unsaponifiable species including glycerol, squalene, waxes, carotenoid pigments, and tocopherols. An inexpensive mineral acid such as sulfuric acid or hydrochloric acid is ideal for this purpose. Once a pH of 4 or less is achieved by the addition of acid, the mixture will separate into two immiscible layers. The bottom aqueous layer contains dissolved potassium salts (potassium sulfate when sulfuric acid is employed; potassium chloride when hydrochloric acid is employed) and glycerol. The supernatant layer contains free fatty acids (liberated from their potassium soaps by the mineral acid), squalene, waxes, carotenoid pigments, and tocopherols.
7.) The supernatant layer containing the tocopherols is removed.
8.) The tocopherols can be recovered in high (>70%) purity and high (>70%) yield by distillation under reduced pressure. After the more volatile free fatty acids are topped off, a “heart cut” containing concentrated tocopherols can be obtained, typically at pot temperatures of 230° C. to 260° C. at pressures of 0.3 to 0.4 mm Hg. On a commercial scale, a short-path molecular still is the ideal apparatus for this purpose.

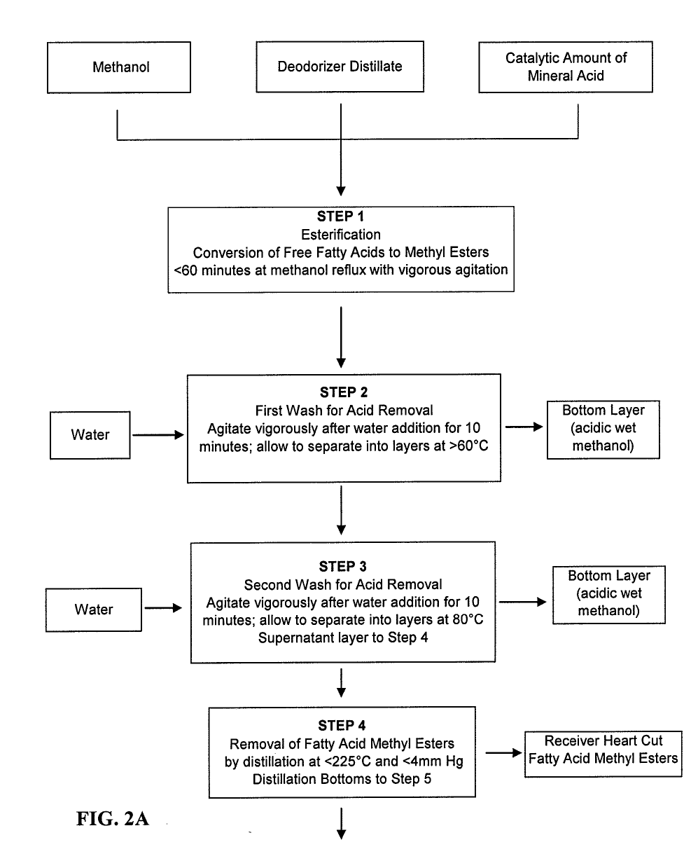

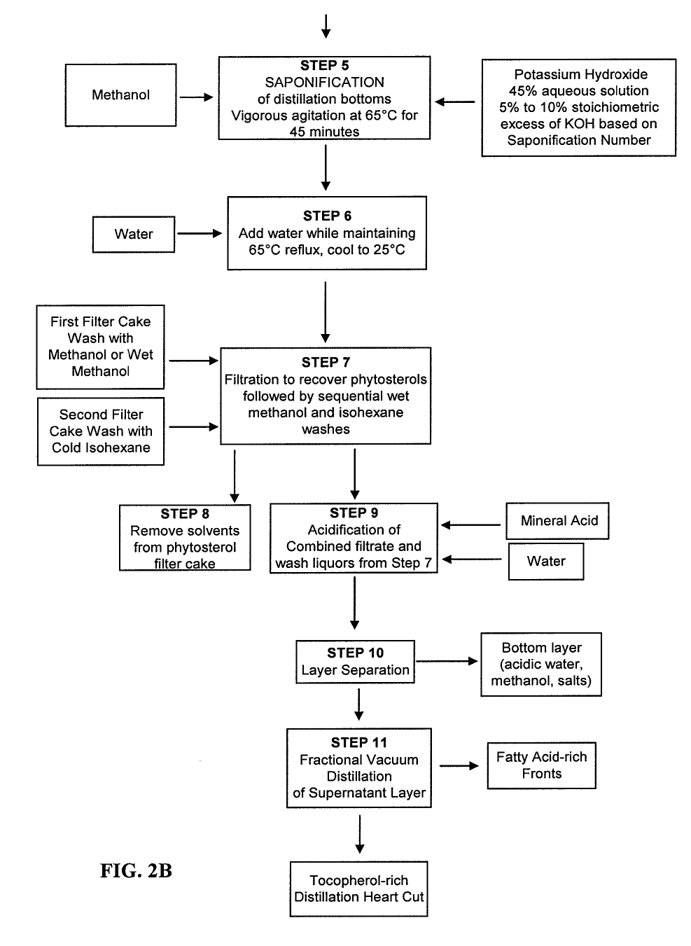

Prior to saponifying the deodorizer distillate, it is often advantageous to employ a process step for the removal of free fatty acids as described in U.S. Pat. Nos. 2,704,764, 3,122,565, 3,153,055, and 3,335,154, which all describe various processes for the isolation of phytosterols from deodorizer distillates. The contents of each of these patents are hereby incorporated by reference. In each of these processes an essential step comprises treating the deodorizer distillate at elevated temperatures with a lower aliphatic alcohol like methanol in the presence of an acid catalyst like sulfuric acid. This process can also be used as an optional step in accordance with certain aspects of the present invention as illustrated in the flow chart in FIG. 2. The acid-catalyzed reaction with a lower aliphatic alcohol results in the esterification of the free fatty acids present. The fatty acid methyl esters that are formed are essentially inert to free phytosterols and tocopherols even at elevated temperatures, and, being considerably more volatile than the corresponding free fatty acids, can be more readily separated by distillation under reduced pressure.

The preliminary removal of the free fatty acids from the deodorizer distillate in this fashion results in a substantial reduction in the amount of alkali that is required to liberate free phytosterols by saponifying the phytosterol esters because the free fatty acids would otherwise have consumed a considerable amount of potassium hydroxide. After the fatty acid methyl esters are removed by distillation, the heel that remains contains essentially all of the phytosterol esters and a portion of the various glyceride esters of fatty acids originally present in the deodorizer distillate, as well as all of the unsaponifiable matter, namely terpenic and aliphatic alcohols, waxes, squalene pigments, free phytosterols, and tocopherols. This heel, which typically has a mass of less than 60% of the deodorizer distillate from which it is derived and a saponification number in the range of 120 to 140, can then be subjected to the process in the same fashion as raw deodorizer distillate. This typically corresponds to a two-fold reduction in the amount of potassium hydroxide required to effect the saponification of the phytosterol esters as well as higher reactor loading during the saponification step.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the present specification are approximations that may vary depending upon the desired properties sought to be obtained by the present invention. The terms “about” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values.

Unless otherwise indicated, all percentages of compositions referred to herein are weight percentages of the total composition (i.e. the sum of all components present) and all ratios are weight ratios.

The present invention is further illustrated by the following non-limiting examples.

### EXAMPLES

### Example 1

A sample of deodorizer distillate derived from the refining of soybean oil was assayed and determined to have a saponification number of 163, a phytosterol content (both in free form and as phytosterol esters, predominantly the latter) of 16.5% by weight, and a tocopherol content of 6.4% by weight. 1,000 grams of this material was added to 3,000 grams of vigorously stirred methanol contained in a 3-necked flask equipped with a heating mantle, paddle stirrer, thermal well, and reflux condenser. After the temperature of the resulting mixture was increased to 55° C., 398 grams of a 45% by weight aqueous solution of potassium hydroxide (containing 179 grams of dissolved potassium hydroxide, a 10% stoichiometric excess of the amount required to neutralize the free fatty acids and to saponify the esters present) was slowly added. The temperature of the reaction mixture was increased to 66° C. while maintaining vigorous stirring. Once this temperature was achieved, the mixture was allowed to reflux for 45 minutes to complete the saponification reaction. 400 grams of de-ionized water were added, for a total of 619 grams of water present (including the 219 grams contained in the aqueous potassium hydroxide solution added previously), thereby creating a solvent consisting of 3,000 grams of methanol, 619 grams of water (corresponding to 83% methanol/17% water), and dissolved potassium soaps.

After restoring reflux for a period of 10 minutes, the mixture was cooled to 24° C. to allow the dissolved phytosterols to crystallize out, then filtered on a Buechner funnel through Whatman #4 (20 micron) paper. Filtration was rapid, with no proclivity to blind.

The filter cake was washed with 380 grams of methanol at ambient temperature (18° C.), followed by a wash with 200 grams of isohexane chilled to 5° C. to minimize the solubility of the phytosterols. The off-white, crystalline filter cake was dried at 80° C. in a vacuum oven to yield 147.9 grams of phytosterols that were assayed by gas chromatography at 93.7% purity, corresponding to a recovery of 84% of the sterols contained in the deodorizer distillate. The tocopherol content of the phytosterols was only 0.14% by weight.

The combined filtrate and wash liquors were combined with one liter of water in a beaker equipped with a magnetic stirring bar and a stirring hot plate. 150 grams of a 50% by weight aqueous solution of sulfuric acid were added with vigorous stirring. The mixture was heated to 40° C. and charged to a separatory funnel. The bottom aqueous layer (pH=3.3) containing excess mineral acid and acidic potassium sulfate was removed and discarded. The dark brown supernatant layer containing free fatty acids and unsaponifiable matter (squalene, carotenoid pigments, waxes, and tocopherols) was dried by heating to 120° C. under reduced pressure, then charged to a small glass molecular still. A fraction rich in fatty acids and squalene was topped off at evaporator temperatures of 165° C. to 180° C. while maintaining a pressure of 0.066 mBar. A “heart cut” fraction rich in tocopherols with a mass of 63 grams was taken off at evaporator temperatures of 220° C. to 240° C. at the same pressure. Analysis of this fraction by gas chromatography gave a 72% by weight tocopherols content and a phytosterols content of only 3.2% by weight. This corresponds to a tocopherol recovery of 71%.

### Example 2

A sample of deodorizer distillate derived from the refining of canola oil was assayed and determined to have a saponification number of 172, a phytosterol content (both in free form and as phytosterol esters, predominantly the latter) of 14.2% by weight, and a tocopherol content of 5.8% by weight. 1,000 grams of this material was added to 2,500 grams of vigorously stirred methanol contained in a 3-necked flask equipped with a heating mantle, paddle stirrer, thermal well, and reflux condenser. After the temperature of the resulting mixture was increased to 55° C., 420 grams of a 45% by weight aqueous solution of potassium hydroxide (containing 189 grams of dissolved potassium hydroxide, a 10% stoichiometric excess of the amount required to neutralize the free fatty acids and to saponify the esters present) was slowly added. The temperature of the reaction mixture was increased to 66° C. while maintaining vigorous stirring. Once this temperature was achieved, the mixture was allowed to reflux for 45 minutes to complete the saponification reaction. 300 grams of de-ionized water were added, for a total of 531 grams of water present (including the 231 grams contained in the aqueous potassium hydroxide solution added previously), thereby creating a solvent consisting of 2,500 grams of methanol, 531 grams of water (corresponding to 82% methanol/18% water), and dissolved potassium soaps.

After restoring reflux for a period of 10 minutes, the mixture was cooled to 22° C. to allow the dissolved phytosterols to crystallize out, then filtered on a Buechner funnel through Whatman #4 (20 micron) paper. Filtration was rapid, with no proclivity to blind.

The filter cake was washed with 350 grams of methanol at ambient temperature (18° C.), followed by a wash with 180 grams of isohexane chilled to 5° C. to minimize the solubility of the phytosterols. The off-white, crystalline filter cake was dried at 80° C. in a vacuum oven to yield 121.6 grams of phytosterols that were assayed by gas chromatography at 94.6% purity, corresponding to a recovery of 81% of the sterols contained in the deodorizer distillate. The tocopherol content of the phytosterols was only 0.20% by weight.

The combined filtrate and wash liquors were combined with 750 milliliters of water in a beaker equipped with a magnetic stirring bar and a stirring hot plate. 150 grams of a 50% by weight aqueous solution of sulfuric acid were added with vigorous stirring. The mixture was heated to 40° C. and charged to a separatory funnel. The bottom aqueous layer (pH=3.1) containing excess mineral acid and acidic potassium sulfate was removed and discarded. Residual water was removed from the dark brown supernatant layer containing free fatty acids and unsaponifiable matter (squalene, carotenoid pigments, waxes, and tocopherols) by heating it to 120° C. under reduced pressure. The dried supernatant layer was then charged to a small glass still equipped with an insulated Vigreau column. A fraction rich in fatty acids and squalene was topped off at pot temperatures less than 250° C. and overhead temperatures less than 227° C. while maintaining pressures of 0.06 to 0.07 mm Hg. A “heart cut” fraction rich in tocopherols with a mass of 54.2 grams was taken off at pot temperatures ranging from 253° C. to 280° C. at the same pressure. Analysis of this fraction by gas chromatography gave a 76% by weight tocopherols content and a phytosterols content of only 4.0% by weight. This corresponds to a tocopherol recovery of 74%.

### Example 3

A sample of deodorizer distillate derived from the refining of soybean oil was assayed and determined to have a saponification number of 163, a phytosterol content (both in free form and as phytosterol esters, predominantly the latter) of 16.5% by weight, and a tocopherol content of 6.4% by weight. One kilogram of this material was added to 500 grams of methanol containing 15 grams of concentrated sulfuric acid and refluxed for 45 minutes with vigorous stirring. Analysis of an aliquot of the mixture was analyzed by gas chromatography revealed a 93% conversion of free fatty acids to their corresponding methyl esters. 100 milliliters of de-ionized water was added and the mixture was agitated vigorously for ten minutes, then charged to a separatory funnel. The bottom aqueous layer containing sulfuric acid was removed and discarded. A second wash using 200 milliliters of de-ionized water was performed to ensure the thorough removal of sulfuric acid.

The fatty acid methyl esters were removed from the supernatant organic layer by distillation under reduced pressure (<4 mm Hg) while taking the pot to a temperature of 220° C. The mass of the fatty acid methyl esters collected in the receiver was 448 grams and contained less than 4% free fatty acids by weight according to gas chromatography analysis.

The bottoms from the distillation had a mass of 547 grams and a saponification number of 132. This material was added to 1,100 grams of stirred methanol contained in a 3-necked flask equipped with a heating mantle, paddle stirrer, thermal well, and reflux condenser. After the temperature of the resulting mixture was increased to 55° C., 178 grams of a 45% by weight aqueous solution of potassium hydroxide (containing 79.9 grams of dissolved potassium hydroxide, a 10% stoichiometric excess of the amount required to neutralize the free fatty acids and to saponify the esters present) was slowly added. Note that following the preliminary conversion of the fatty acids contained in the deodorizer distillate to their corresponding methyl esters, which were then removed by fractional distillation, the amount of potassium hydroxide required to saponify the phytosterol- and tocopherol-containing fraction was reduced by a factor of 2.2 compared to Example 1. In addition, the amount of methanol required to render soluble the soaps and unsaponifiables was also reduced.

The temperature of the reaction mixture was increased to 66° C. while maintaining vigorous stirring. Once this temperature was achieved, the mixture was allowed to reflux for 45 minutes to complete the saponification reaction. 270 grams of de-ionized water were added, for a total of 368 grams of water present (including the 98 grams contained in the aqueous potassium hydroxide solution added previously), thereby creating a solvent consisting of 1,100 grams of methanol, 368 grams of water (corresponding to 75% methanol/25% water), and dissolved potassium soaps.

After restoring reflux for a period of 10 minutes, the mixture was cooled to 22° C. to allow the dissolved phytosterols to crystallize out, then filtered on a Buechner funnel through Whatman #4 (20 micron) paper. Filtration was rapid, with no proclivity to blind.

The filter cake was washed with 385 grams of methanol at ambient temperature (18° C.), followed by a wash with 200 grams of isohexane chilled to 5° C. to minimize the solubility of the phytosterols. The off-white, crystalline filter cake was dried at 80° C. in a vacuum oven to yield 144.8 grams of phytosterols that were assayed by gas chromatography at 95.1% purity, corresponding to a recovery of 83% of the sterols contained in the deodorizer distillate. The tocopherol content of the phytosterols was only 0.11% by weight.

The combined filtrate and wash liquors were combined with one liter of water in a beaker equipped with a magnetic stirring bar and a stirring hot plate. 150 grams of a 50% by weight aqueous solution of sulfuric acid were added with vigorous stirring. The mixture was heated to 40° C. and charged to a separatory funnel. The bottom aqueous layer (pH=3.3) containing excess mineral acid and acidic potassium sulfate was removed and discarded. Residual water was removed from the dark brown supernatant layer containing free fatty acids and unsaponifiable matter (squalene, carotenoid pigments, waxes, and tocopherols) by heating it to 120° C. under reduced pressure. The dried supernatant layer was then charged to a small glass still equipped with an insulated Vigreau column. A fraction rich in fatty acids and squalene was topped off at pot temperatures less than 250° C. and overhead temperatures less than 227° C. while maintaining pressures of 0.06 to 0.07 mm Hg. A “heart cut” fraction rich in tocopherols with a mass of 66 grams was taken off at pot temperatures ranging from 253° C. to 280° C. at the same pressure. Analysis of this fraction by gas chromatography gave a 74% by weight tocopherols content and a phytosterols content of only 3.2% by weight. This corresponds to a tocopherol recovery of 76%.

What is claimed is:

1. A process for recovering phytosterols from deodorizer distillate comprising:
providing a deodorizer distillate composition wherein said composition comprises phytosterols, esters, and fatty acids;
saponifying the esters and fatty acids in the deodorizer distillate composition by contacting the deodorizer distillate composition with a first lower aliphatic alcohol and potassium hydroxide; and
adding water to form a mixture comprising a precipitate containing the phytosterols.

2. The process of claim 1 further comprising recovering the phytosterols by filtration or centrifugation.

3. The process of claim 1 wherein the saponification reaction is conducted at a temperature from about 50° C. to about 160° C.

4. The process of claim 3 further comprising cooling the mixture to a temperature of about 15° to about 30° C. after the saponification reaction is complete.

5. The process of claim 2 further comprising washing the recovered phytosterols with a solution comprising a methanol or a mixture of a methanol and water to provide a first phytosterol product having a purity of more than about 88%.

6. The process of claim 5 further comprising washing the first phytosterol product with a non-polar solvent to provide a second phytosterol product having a purity of more than about 95%.

7. The process of claim 1 further comprising treating the deodorizer distillate with a second lower aliphatic alcohol in the presence of an acid catalyst to esterify free fatty acids and then removing the free fatty acids in the form of esters by distillation before the saponification step.

8. The process of claim 7 wherein the first and second lower aliphatic alcohols are methanol.

9. A process for recovering phytosterols and/or tocopherols from deodorizer distillate comprising:
providing a deodorizer distillate composition wherein said composition comprises fatty acid esters of phytosterols and free tocopherols;
contacting the deodorizer distillate composition with methanol and potassium hydroxide to saponify the fatty acid esters of phytosterols contained in the deodorizer distillate composition to form a saponification reaction mixture; and
adding water to the saponification reaction mixture to form a solvent containing methanol, water, and the potassium soaps of fatty acids wherein the solvent causes the phytosterols to precipitate.

10. The process of claim 9 further comprising recovering the phytosterols by filtration or centrifugation.

11. The process of claim 9 wherein the saponification reaction is conducted at a temperature from about 50° C. to about 160° C.

12. The process of claim 9 further comprising cooling the mixture to a temperature of about 15° to about 30° C. after the saponification reaction is complete.

13. The process of claim 9 wherein the amount of water in the solvent falls in the range of about 5% to 30% by weight based on the total weight of water and methanol in the solvent.

14. The process of claim 9 wherein the amount of the potassium soaps of fatty acids in the solvent is in the range of about 4% to 25% by weight based on the total weight of the solvent.

15. The process of claim 10 further comprising washing the recovered phytosterols with a wash solution comprising methanol or a mixture of methanol and water to provide an intermediate phytosterol product.

16. The process of claim 15 wherein the ratio of water to methanol in the wash solution ranges from about 5% to 25% by weight.

17. The process of claim 15 wherein the intermediate phytosterol product has a purity of more than about 85%.

18. The process of claim 15 further comprising washing the intermediate phytosterol product with a non-polar solvent to provide a second phytosterol product of higher purity.

19. The process of claim 18 wherein the second phytosterol product has a purity of more than about 90%.

20. The process of claim 15 wherein the non-polar washing solvent is selected from the group consisting of isohexane, n-hexane, hexane isomers, n-heptane, heptane isomers and mixtures thereof.

21. The process of claim 15 wherein the non-polar washing solvent is cooled to less than ambient temperature.

22. The process of claim 10 wherein the solvent dissolves or renders miscible the tocopherols and other unsaponifiable matter such that the tocopherols and other unsaponifiable matter predominantly remain in the centrate or filtrate and the tocopherols are isolated from the filtrate or centrate by acidifying with a dilute aqueous solution of mineral acid to form a water-immiscible mixture of free fatty acids, tocopherols, and other unsaponifiable matter, separating the water-immiscible mixture of free fatty acids, tocopherols, and other unsaponifiable matter and isolating the tocopherols by fractional distillation under reduced pressure.

23. The process of claim 22 wherein the mineral acid is sulfuric acid.

24. The process of claim 22 wherein the mineral acid is hydrochloric acid.

25. A process for treating deodorizer distillate to facilitate recovery of phytosterols and/or tocopherols from the deodorizer distillate comprising:
contacting a deodorizer distillate composition comprising fatty acid esters of phytosterols and free tocopherols with methanol and potassium hydroxide to saponify the fatty acid esters of phytosterols contained in the deodorizer distillate composition to form a saponification reaction mixture; and
adding water to the saponification reaction mixture to form a solvent containing methanol, water, and the potassium soaps of fatty acids wherein the tocopherols are soluble or miscible in the solvent and the phytosterols are insoluble in the solvent, thereby forming a precipitate containing the phytosterols.

26. The process of claim 25 wherein the amount of water in the solvent falls in the range of about 5% to 30% by weight based on the total weight of water and methanol in the solvent.

27. The process of claim 25 wherein the amount of the potassium soaps of fatty acids in the solvent is in the range of about 4% to 25% by weight based on the total weight of the solvent.
